(19) United States
(12) Patent Application Publication
(10) Pub. No.: US 2026/0165783 A1
(43) Pub. Date: Jun. 18, 2026

(54) INTERACTIVE SIMULATIONS OF HUMAN BODY WITH AUGMENTED REALITY INTEGRATION

(71) Applicant: University of Hartford, West Hartford, CT (US)

(72) Inventor: Dan Liu, Madison, CT (US)

(21) Appl. No.: 18/980,578

(22) Filed: Dec. 13, 2024

(51) Int. Cl. A61B 34/00; A61B 34/20; A61B 90/00

(52) U.S. Cl. CPC A61B 34/00; A61B 2034/2055; A61B 2034/258; A61B 2090/365

(57) ABSTRACT

A method of calibrating an augmented reality system. The method includes outputting first instructions that can communicate to a subject to position their body in a first position. The method also includes obtaining a first measurement with a single sensor and communicating the first measurement to a processor. The method further includes outputting second instructions that can communicate to a subject to position their body in a second position. The method also includes obtaining a second measurement with the single sensor and communicating the second measurement to the processor. Finally, the method includes calculating a length based on the first measurement and the second measurement.

### FIELD

The present disclosure relates to augmented reality. More particularly, the present disclosure relates to an augmented reality system for modeling static and dynamic forces on a subject.

### BACKGROUND

Augmented reality (AR) is an interactive, computer-generated experience that modifies or changes a user's real-world experience by incorporating digital content. In some examples, the digital content may be overlayed onto a user's real-life experience so that their observations in the real world are combined with the computer-generated content to alter the user's perception of the real-world environment.

Biomechanics may use AR simulations of the human body to provide a user with an interactive experience. For example, the simulations may provide information related to forces on a user's muscles and/or joints based on user measurements. These computer-generated outputs may be used to assist athletes in their training regimen and/or can be used to assist in treating a patient (e.g., physical therapy, rehabilitation training, etc.).

Many current biomechanical AR simulations require a user to wear a number of sensors across their body. Each sensor may measure a specific portion of the user's body. The information may be collected and analyzed by a computer to produce a computer-generated output. While this method may be effective in measuring a user's body, it may be expensive to utilize a vast number of sensors and may therefore be prohibitive to a wide audience of potential users.

Existing biomechanical AR simulations may measure static forces on a subject but may be unable to capture dynamic forces on a subject because existing simulation algorithms are too complex or slow to analyze and process real-time video. In other words, an existing AR simulation may be unable to measure forces on a user in real time as the user moves and needs to re-analyze the forced experienced by the user at discrete positions. Models that measure only static forces provide an incomplete picture of the biomechanical forces experienced by the user.

Some models that can measure dynamic forces. However, the dynamic measurements are generally made on a pre-recorded video. Subjects of the video recordings therefore cannot receive instant, real-time feedback.

A need exists for an AR simulation that is able to measure the forces on a user's muscles and joints both statically and dynamically, while not being cost prohibitive by requiring wearable sensors across the body.

### SUMMARY

Various embodiments of the present disclosure can overcome various of the aforementioned and other disadvantages associated with known augmented reality systems and offer new advantages as well.

According to one aspect of various embodiments of the present disclosure there is provided augmented reality system capable of measuring static and dynamic forces on the human body.

According to one aspect of various embodiments of the present disclosure there is provided augmented reality system that includes a sensor that can measure the static and/or dynamic forces on a human body.

According to another aspect of various embodiments of the present disclosure, there is provided a system that includes a computer, a sensor, and a display.

According to another aspect of various embodiments of the present disclosure, there is provided an augmented reality system that includes a computer with a processor, an imaging sensor connected to the computer, and a display connected to the computer. The imaging sensor can transmit measurements to the computer. The display can output data from the computer. The computer can implement a diagnostic program to calculate a size of a human subject. The diagnostic program includes outputting an initial instruction via the display that can instruct a subject to stand an initial distance from the imaging sensor. The diagnostic program also includes outputting a first calibration instruction via the display that can instruct the subject to orient their arms in a first position. The diagnostic program also includes receiving, from the imaging sensor, a first measurement following delivery of the first calibration instruction. The diagnostic program further includes outputting a second calibration instruction via the display configured to instruct the subject to orient their arms in a second position. The diagnostic program also includes receiving, from the imaging sensor, a second measurement following delivery of the second calibration instruction. The diagnostic program also includes calculating the depth of a body of the human subject, using the first measurement and the second measurement

According to another aspect of various embodiments of the present disclosure, there is provided a method of calibrating an augmented reality system. The method includes orienting a single optical sensor in a first direction. The single optical sensor is connected to a computer and includes a line of sight. The method also includes outputting first instructions via a display connected to the computer. The first instructions can communicate to a subject to move into the line of sight and position their body in a first position. The method also includes obtaining a first measurement with the optical sensor. The first measurement is a first length of a first segment of the body. The method further includes communicating the first measurement to a processor of the computer and outputting second instructions via the display. The second instructions can communicate to move into the line of sight and a subject to position their body in a second position. The method also includes obtaining a second measurement with the optical sensor. The second measurement is a second length of the first segment of the body. The method further includes communicating the second measurement to the processor and calculating, via the processor, a depth based on the first measurement and the second measurement. Calculating includes determining the value of a first constant using the first measurement and the second measurement, determining the value of a second constant using the first measurement and the second measurement, determining the distance using the first constant and the second constant, and storing the distance in the computer as the depth.

According to another aspect of various embodiments of the present disclosure, there is provided a method of calibrating an augmented reality system.

According to another aspect of various embodiments of the present disclosure, there is provided a method of calibrating an augmented reality system. The method includes outputting first instructions that can communicate to a subject to position their body in a first position. The method also includes obtaining a first measurement with a single sensor and communicating the first measurement to a processor. The method further includes outputting second instructions that can communicate to a subject to position their body in a second position. The method also includes obtaining a second measurement with the single sensor and communicating the second measurement to the processor. Finally, the method includes calculating depth based on the first measurement and the second measurement.

According to another aspect of various embodiments of the present disclosure, there is provided a method of calculating real-time forces experienced by a subject. The method includes calibrating an augmented reality system to a specific subject and calculating the force experienced by the subject once per period. Calibrating includes outputting first instructions via a display connected to the computer. The first instructions can communicate to a subject to move into a line of sight of a sensor and position their body in a first position. Calibrating also includes obtaining a first measurement with the sensor. The first measurement is a first length of a first segment of the body. Calibrating further includes communicating the first measurement to a processor of the computer and outputting second instructions via the display. The second instructions can communicate to move into the line of sight and a subject to position their body in a second position. Calibrating also includes obtaining a second measurement with the sensor. The second measurement is a second length of the first segment of the body. Calibrating further includes communicating the second measurement to the processor and calculating, via the processor, a depth based on the first measurement and the second measurement. Calculating includes measuring a first orientation angle of the first segment using the sensor and communicating the first orientation angle to the computer. Calculating also includes calculating a first lever arm length using the first orientation angle, determining a first force using the first lever arm length, and outputting the first force on the display.

According to another aspect of various embodiments of the present disclosure, there is provided an augmented reality system that includes a computer with a processor, an imaging sensor connected to the computer, and a display connected to the computer. The imaging sensor can transmit measurements to the computer. The display can output data from the computer. The computer can implement a diagnostic program to calculate a size of a human subject. The diagnostic program includes outputting an initial instruction via the display that can instruct a subject to stand an initial distance from the imaging sensor. The diagnostic program further includes orienting the imaging sensor in a first direction. The imaging sensor includes a line of sight. The diagnostic program also includes outputting a first calibration instruction via the display that can instruct the subject to move into the line of sight and orient their arms in a first position. The diagnostic program also includes receiving, from the imaging sensor, a first measurement following delivery of the first calibration instruction. The first measurement is a first length of a first segment of the body. The diagnostic program also includes outputting a second calibration instruction via the display configured to instruct the subject to move into the line of sight and orient their arms in a second position. The diagnostic program also includes receiving, from the imaging sensor, a second measurement following delivery of the second calibration instruction. The first measurement is a first length of a first segment of the body. The diagnostic program further includes calculating, using the first measurement and the second measurement, a depth. The calculating includes determining a value of a first constant using the first measurement and the second measurement, determining a value of a second constant using the first measurement and the second measurement, determining the distance using the first constant and the second constant, and storing the distance in the computer as the depth.

The disclosure herein should become evident to a person of ordinary skill in the art given the following enabling description and drawings. The drawings are for illustration purposes only and are not drawn to scale unless otherwise indicated. The drawings are not intended to limit the scope of the invention. The following enabling disclosure is directed to one of ordinary skill in the art and presupposes that those aspects within the ability of the ordinarily skilled artisan are understood and appreciated.

### BRIEF DESCRIPTION OF THE DRAWINGS

Various aspects and advantageous features of the present disclosure will become more apparent to those of ordinary skill when described in the detailed description of preferred embodiments and reference to the accompanying drawing wherein:

FIG. 1 is a schematic view of a biomechanical AR system.

FIG. 2 is a schematic view of a graphical user interface on a display of the AR system of FIG. 1.

FIG. 3 is a flow chart illustrating the calibration process.

FIG. 4-1 is a perspective view of a subject in a first calibration position.

FIG. 4-2 is a perspective view of a subject in a second calibration position.

FIG. 4-3 is a perspective view of a subject in a third calibration position.

FIG. 5 is a schematic view of a force diagram on a subject for use in calculating a real-time force.

### DETAILED DESCRIPTION

The biomechanical AR system may be an interactive integration of AR technology that produces simulations of the human body to provide an in-time interactive user experience. The system may follow movements of the user to provide real-time quantitative measurements and analyses for the forces on the muscles and/or joints in real-life environmental settings.

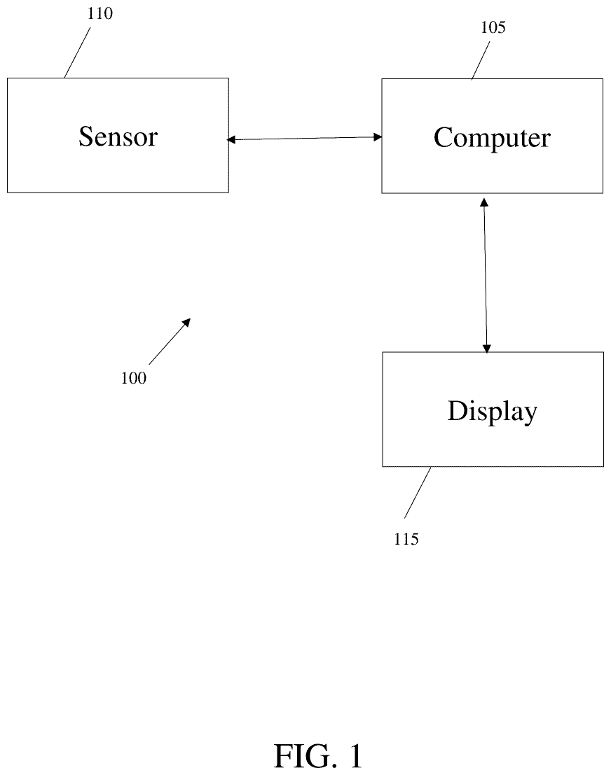

As shown in FIG. 1, the biomechanical AR system 100 may include a computer 105, a sensor 110, and a display 115. The computer 105 may be in communication with the sensor 110 and the display 115 to send and/or receive inputs and outputs. The communication link between the computer 105 and the sensor 110 and/or display 115 may be a wired connection and/or a wireless connection (e.g., via Bluetooth, Wi-Fi, cellular, etc.).

In some forms, the sensor 110 may be a single sensor. For example, the sensor 110 may be an image-capture sensor, like a camera (e.g., a computer webcam). The sensor 110 may be able to take videos and/or photos of a subject (e.g., a human subject) and transmit the captured data (either wirelessly or via a wired connection) to the computer 105. As described in more detail below, the computer 105 may use the data from the videos and/or photos collected by the sensor 110 to determine static and/or dynamic forces on a human subject.

In other examples, multiple sensors 110 (e.g., two, three, etc.) may be in communication with the computer 105. For example, the two or more sensors 110 may be multiple image-capture sensors connected to the computer. The multiple image-capture sensors 110 may be used to capture multiple angles of a single subject and/or to simultaneously track multiple subjects. In still other examples, the multiple sensors 110 could include a single image-capture sensor 110 and one or more other types of sensors (e.g., a heat sensor, a motion sensor, a microphone, etc.). The one or more other types of sensors can collect additional data that can be used by the computer 105. For example, the additional sensors can measure and analyze forces experienced by the subject.

In some forms, the display 115 may be a computer monitor or any other similar output device (e.g., a smart phone) that can visually display data to a user. The display 115 may include graphical user interfaces (GUIs) that may permit the user to interact with the computer 105 through the display 115. For example, the user may interact with the display 115 to enter inputs and/or receive outputs from the computer 105. Other examples may include different types of audio or visual indicators that can communicate information (e.g., LEDs, speakers, etc.).

A user many interact with the computer 105, the sensor 110, and/or the display 115 of the AR system 100 to create a computer-generated output. To create the output, the computer 105 may receive input data that can be collected via the sensor 110 and/or via a user input.

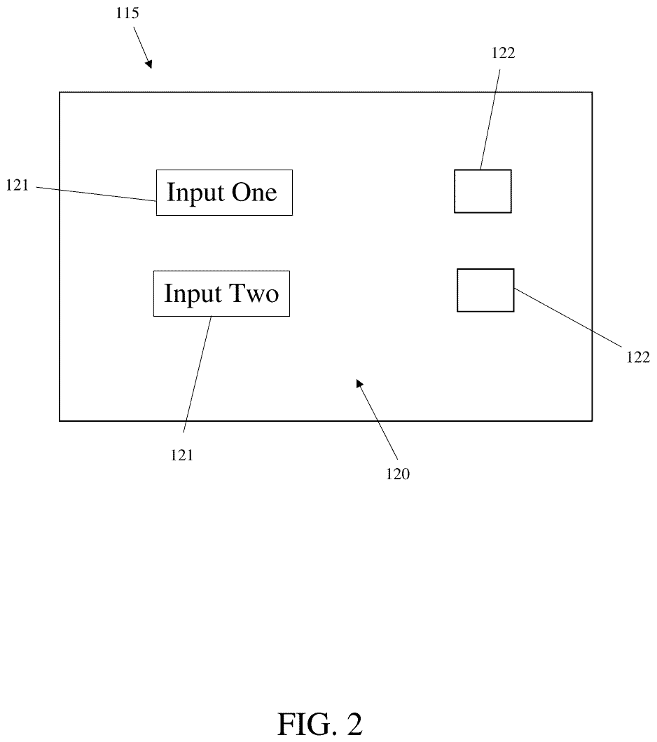

As shown in FIG. 2, the display 115 may include a GUI that can receive user-inputted data. For example, the display may include one or more data request fields 121 and one or more data entry fields 122 (e.g., two shown for each). The data request fields 121 may prompt a person to enter specific types of data (e.g., subject height) and the corresponding data entry field 122 may provide the person space to enter data requested by the computer 105. Data entered into the data entry field 122 may be communicated to the computer 105 for use in its calculations.

In some forms, the GUI may be a calibration screen 120 that can assist in producing a computer-generated output. For example, the computer 105 may be calibrated to a particular subject to analyze the data in relation to that particular subject. Using one or more sensors 110 not worn by the subject may use additional inputs to correctly measure the movements of the subject.

In certain forms, a first step of the calibration process may be entering computer-requested measurements on the calibration screen. In the illustrated example, a user can enter one or more of the subject's height, the subject's weight, the distance between the subject and the sensor 115, and the weight of an object held by the subject. In other examples, one or more of these measurements may be excluded and/or the calibration screen 120 may include the ability to add additional information.

A user may enter the requested values (e.g., via a keyboard, a touch screen, etc.) on the calibration screen of the display 115. As described in more detail below, the values may assist the sensor in calibrating to or measuring the subject. Once the values are entered, the data is communicated to a processor in the computer 105 and the calibration process can continue.

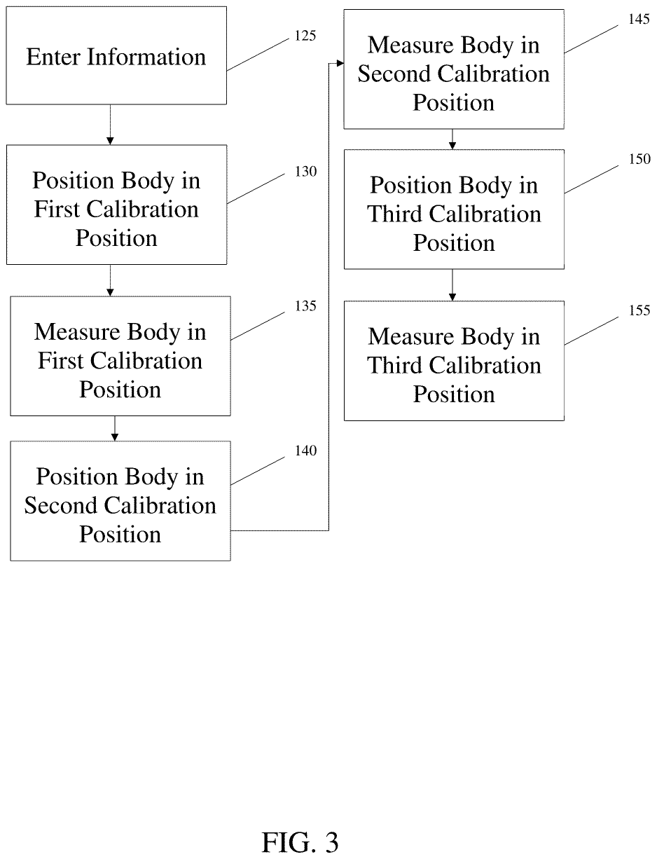

Once the requested values are entered on the calibration screen 120, the calibration process may continue by collecting additional data about the subject. As illustrated in FIG. 3, once the requested values are communicated to the processor and the first calibration step 125 is complete, the computer 105 may prompt the subject to perform additional calibration steps. These additional calibration steps may include evaluating the subject in one or more different positions to calculate and verify measurements of the subject. The illustrated example includes three separate calibration positions, although other examples could include a different number.

In particular, the computer 105 may prompt the subject (e.g., through the display 115) to orient their body in one or more pre-selected positions. These positions may enable the sensor 110 to take measurements of the subject. These measurements from the sensor 110 may be combined with entries from the calibration screen 120 to permit the computer 105 to create a profile on the subject.

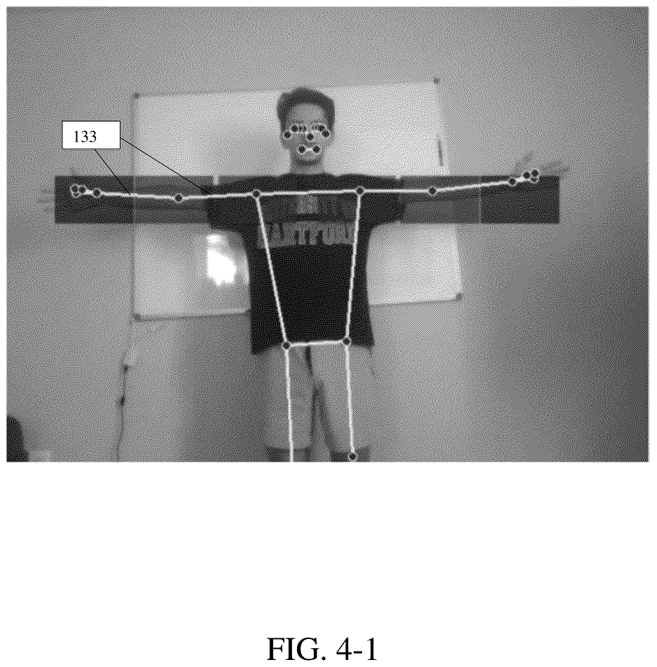

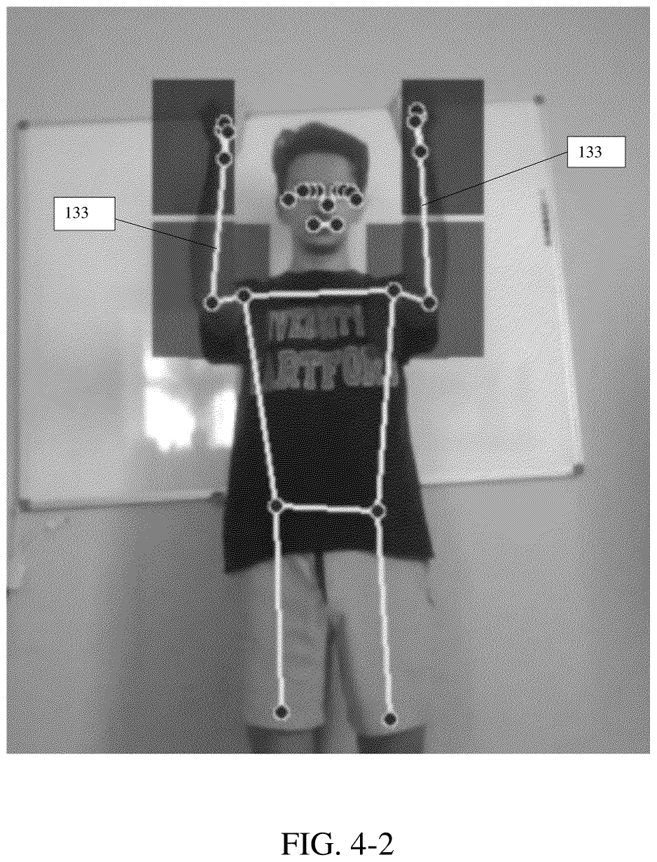

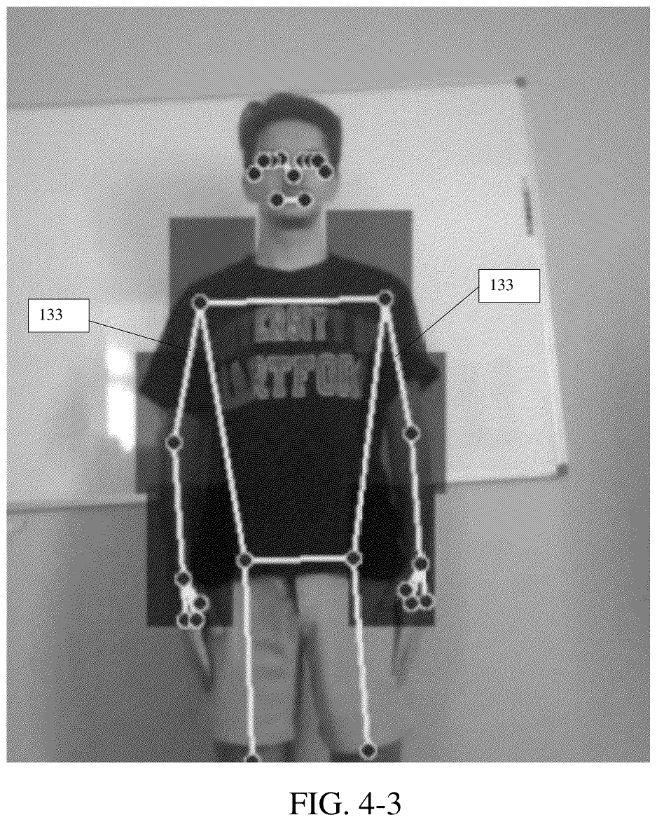

In one form, the computer 105, via the display 115, may prompt 130 the subject to orient their body in a first position. As illustrated in FIG. 4-1, the subject may be prompted (e.g., via an output from the display 115) to stand with their arms spread out (e.g., with the subject's arms oriented at the full wingspan about 180 degrees apart). While in the first calibration position 130, the sensor 110 may measure 135 and collect data from the subject.

In some forms, the subject may stand far enough from the sensor 110 so that their entire wingspan is in frame (e.g., where the camera angle is wider than the subject's maximum wingspan). The computer 105, via the display 115, may prompt the subject to stand a pre-specified distance from the sensor 110 (e.g., about 3 feet) where the subject's wingspan will be fully captured by the sensor 110. Once the subject has positioned themselves a certain distance from the sensor 110, the display 115 may prompt the subject to remain in that location (e.g., refrain from moving closer to or further from the sensor 110).

The image-capture sensor 110 may be focused on the body of the subject. When the subject has oriented their body in the first calibration position, the sensor 110 may make various measurements 135 of the subject's body in the first position. For example, the sensor 110 may detect various segments 133 of the subject's body and the computer 105 can calculate the length of each segment. These measurements may be communicated to the processor of the computer 105 to begin calibrating the system 100 to the individual subject.

In some forms, the computer 105 may calculate a calibration ratio when the subject has oriented their body in the first position. As described in more detail below, the calibration ratio may be used to adjust an initial length calculation by the computer 105 to obtain a more accurate measurement. The calibration ratio may be based on the subject's height, which was entered by the subject (or another person) in step 125, and an arm span length, which may be determined by the computer 105 (e.g., using measurements taken by the sensor 110 while the subject is in the first calibration position). The calibration ratio R has the following form where the subject's height HP is directly proportional to R and the subject's arm span LAS as measured by the computer 105 and sensor 110 is inversely proportional to R.

$$R = {H}_{P} / {L}_{AS} \qquad ( 1 )$$

Using this ratio, the computer 105 can attempt to determine the length of other parts of the subject's body. As illustrated in FIG. 4-1, the computer 105, using the sensor 110, may break the subject's body into discrete segments 133 (e.g., which may correspond to a bone or other unit of the subject's body). For example, the computer 105 can use the calculated ratio and a series of pre-programmed values to determine the length of each segment 133. When measuring the subject's upper body (e.g., as in FIG. 4-1), the subject's body may be broken down into segments 133 that approximate the length of the subject's radius/ulna, humerus, and clavicles.

While these values may provide an estimated length of each segment 133, they are generally inaccurate approximations. Particularly, because while the subject is directed to remain the same distance from the sensor 110 as they entered in step 125, their entire body may not remain at that distance. For example, while the subject's feet may remain stationary, other parts of the subject's body may move to points greater than and/or less than the entered distance. Additionally, the subject might not have initially positioned their body the exact distance requested by the display 115. The measurements could also be inaccurate because an object may appear distorted when it approaches an optical sensor 110 (e.g., an object measured by an optical sensor 110 may appear larger than it is as it moves closer to the optical sensor 110 or smaller as it moves away from the optical sensor 110). Therefore, the computer 105 must continue to calibrate for a particular subject to more accurately identify the length of each segment 133.

In one form, the computer 105, via the display 115, may prompt 140 the subject to orient their body in a second position once the measurements from the first position are recorded. As illustrated in FIG. 4-2, the subject may be prompted to stand with their arms in front of their face, with each elbow bent at about 90 degrees. In this position, at least a portion of the subject's arms are moved closer to the sensor 110 (e.g., even while the subject's feet remain stationary). While in the second calibration position, the sensor 110 may measure 145 and collect data from the subject.

For example, the image-capture sensor 110 may be focused on the body of the subject. When the subject has oriented their body in the second calibration position, the sensor 110 may make various measurements of the subject's body. These measurements may be communicated to the processor of the computer 105 to continue calibrating the system 100 to the individual subject.

In particular, the computer 105 may continue measuring the subject to determine a depth of the subject relative to the sensor 110. As described above, although the subject was instructed to stand a specified distance away, the subject may not be that exact distance. Even slight deviations from the originally specified distance can distort the length calculation of the segments 133 conducted in the first measuring step 135. Additionally, the computer 105 may calculate a depth of a various segment 133 relative to the sensor 110 as the subject moves relative to their initial position. For example, portions of the subject's body may move closer to the sensor 110 despite the subject's feet remaining fixed. In a second portion of the calibration step 140, the computer may compare 145 the distance of various segments 133 to the sensor 110 relative to the initially entered distance in step 125 to more accurately determine the size of each segment.

The depth z of a specific body part can be calculated using Equation 2, where Lₘ is the length of the specific segment 133 calculated by the computer 105 when the subject's body was in the first position (see e.g., FIG. 4-1) and L_{c} is the component of Lₘ, measured in a plane perpendicular to the axis of depth Z.

$$z = \sqrt{( {L}_{m}^{2} - {L}_{c}^{2} )} \qquad ( 2 )$$

The component length L_{c} can be calculated using Equation 3, which is a length Lₛ of the segment in display, multiplied by the ratio R calculated in Equation 1.

$${L}_{c} = {L}_{s} \times R \qquad ( 3 )$$

The value of the segment length Ls can be calculated using Equation 4, where d is the subject's initial distance from the sensor 110 and k is a constant.

$${L}_{s} = k / ( d - z ) \qquad ( 4 )$$

When the subject is directed to orient their body in the first position (see e.g., FIG. 4-1), that distance d should remain constant during use as the subject is instructed to remain stationary. This distance d relative to the sensor 110 may be approximately equal to a computer-instructed distance (e.g., assuming that the subject correctly approximated their distance from the sensor 110). However, the depth value may change as the subject moves into the orientation of FIG. 4-2. Specifically, the position of the subject's arms has changed and may be closer to the sensor 110 when they are in the second position 140.

A coordinate plane may be oriented with an origin on the subject when in the first position and a positive value may move in the direction of the sensor 110. Therefore, the length Lₛ of the segment 133 may be equal to the ratio of k/d when the subject is in the first position (e.g., and has not moved toward or away from the sensor 110).

The values of k and d may be based on the length of the subject's forearm (e.g., wrist to elbow), although the computer 105 could use the length of any segment 133. More specifically, the sensor 110 and computer 105 may determine the length of the forearm in the first position (i.e., f_{MP,1}), the length of upper arm in reality (i.e., u) and the length of the forearm in the second position (i.e., f_{MP,2}). The values of k and d can be determined by the equations below.

$$k = - ( {f}_{MP , 1} {f}_{MP , 2} u ) / ( {f}_{MP , 1} - {f}_{MP , 2} ) \qquad ( 5 )$$

$$d = - ( {f}_{MP , 2} u ) / ( {f}_{MP , 1} - {f}_{MP , 2} ) \qquad ( 6 )$$

When a portion of the subject's body moves toward the sensor 110, an enlargement ratio of the segment 133 (e.g., the forearm) may be calculated using the following equation.

$$\frac{{f}_{MP}}{{f}_{MP , 1}} = \frac{{L}_{MP , c} R}{\sqrt{{L}_{m}^{2} - {z}^{2}}} \qquad ( 7 )$$

Equation (7) and Equation (4) can be combined to solve for z in Equation (8) below where the value of a is given in Equation (9).

$$z = \frac{d + a \sqrt{- {d}^{2} + ( 1 + {a}^{2} ) {L}_{m}^{2}}}{1 + {a}^{2}} , \qquad ( 8 )$$

$$where a = \frac{k}{{L}_{MP , c} {Rf}_{MP , 1}} . \qquad ( 9 )$$

This this equation, the value of z may be equal to a coordinate position of the subject's relevant body part. The value of Lₘ may therefore vary based on the specific relevant body part. For example, when attempting to find the z coordinate for the subject's hand, the value of Lₘ may equal the length of the subject's forearm and the value of L_{MP,c} may equal the component of the subject's forearm on display 115. The calculations 145 performed while the subject is in the second position 140 may provide more accurate values over the measurements 135 taken after the first calibration position 130.

In one form, the computer 105, via the display 115, may prompt 150 the subject to orient their body in a third position once the measurements from the first and/or second positions are recorded. As illustrated in FIG. 4-3, the subject may be prompted (e.g., via an output on the display 115) to stand with their arms vertically downwardly, with each with each hand proximate to the respective hip. While in the third position, the sensor 110 may measure 155 and collect data from the subject.

For example, the image-capture sensor 110 may be focused on the body of the subject. When the subject has oriented their body in the third position, the sensor 110 may make various measurements of the subject's body in the third position. These measurements may be communicated to the processor of the computer 105 to complete the calibration the system 100 to the individual subject.

In some forms, directing the subject to orient their body in a third position may reduce distortion in a measurement by the sensor 110 to allow for more accurate measurements. For example, an optical sensor 110 may be set up to measure the subject in a position that is not parallel to the ground. In other words, the optical sensor 110 could be angled relative to the ground (e.g., upwardly or downwardly), which can distort the resulting image that the sensor 110 measures, and thus lead to imprecise measurements of the various segments 133.

Specifically, the orientation of the subject's arms in the position of FIG. 4-3 is substantially opposite the prior position illustrated in FIG. 4-2 (e.g., arms oriented upwardly versus downwardly). Orienting the arms in the final position can reduce or eliminate distortion than can occur from the use of a single sensor 110 so that the resulting value is more accurate. Specifically, the computer 105 compares the previously calculated 135 length values in the first position 130 with the length values calculated 155 in the third position 150 (e.g., comparing the lengths when the subject's arm is oriented in a horizontal and vertical position) to obtain a distortion ratio, which can be used to more accurately determine the length of the different segments 133 irrespective of the angular orientation of the single sensor 110.

In some forms, the subject may be prompted to orient their body in or more additional positions to permit the sensor to collect additional measurements. In other forms, the subject may be prompted to assume one or more alternate positions during the calibration process. In other forms, the subject may be prompted to assume fewer than three positions during the calibration process.

Once the subject as assumed the prompted positions, the computer 105 may be calibrated to the specific individual. The calibration process measures the subject and the environment to enable static and/or dynamic calculations on the subject. In particular, the calibration process may enable the computer 105 to calculate the relative measurements of the subject and the depth of various body parts of the subject. The computer determining this information enables a single sensor 110 to make the calculations.

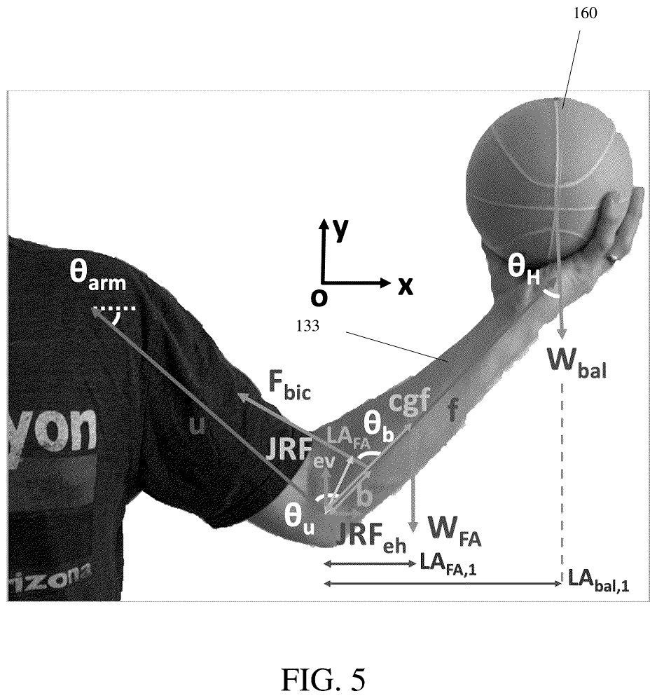

As shown in FIG. 5, forces on a subject may be measured by the sensor after the calibration process is complete. For example, a subject may perform various movements while in range of the sensor 110. As these movements are performed, the calibrated sensor 110 may track the movements of the subject. The sensor 110 can then communicate this information to the computer 105.

In some forms, the calibration steps described above may relate to a specific muscle or muscle group of the subject. For example, the above-described calibration steps may relate to force tracking in an arm of the subject. Thus, the forces shown in FIG. 4 may therefore relate specifically to forces experienced in the subject's arm. The calibration steps performed by the subject may be sufficient to track forces in other muscle groups, or additional and similar calibration steps may be needed when attempting to determine forces in other muscle groups (e.g., a subject's legs).

In some forms, the measurements taken by the sensor 110 may be transmitted to the computer 105, where a force calculation is made. That calculation may then be output to the display 115. For example, the display 115 may be a screen that shows a video feed from the sensor 110. Force calculations may be overlayed onto the subject's body on the display 115.

With continued reference to FIG. 5, the computer 105 may use the measurements taken by the sensor 110 (e.g., the length of one or more segments of the subject's body) to calculate a force on the subject's body. For example, the subject in FIG. 5 may be holding a mass 160. The subject's bicep may apply a force F_{bic} represented for simplicity as extending from the bicep insertion toward the subject's shoulder.

When calculating the force for a static subject, the net force and the net torque are both equal to zero (e.g., the system is at equilibrium). Equations 10 may be used to determine the related forces.

$$0 = {τ}_{bic} - {τ}_{FA , 1} - {τ}_{bal , 1} \qquad ( 10 )$$

In Equation 10, the moment from the force exerted by the bicep (τ_{bic}) is equal to the combined moments resulting from the weight of the subject's forearm (τ_{FA,1}) and the weight of the mass 160 (τ_{bal,1}). Specifically, the resulting force from each of the bicep (F_{bic}), mass 160 (W_{bal}) and forearm (W_{FA}) may be multiplied by their respective lever arms (LA_{bic,1}, LA_{bal,1}, LA_{FA,1})

When the plane defined by the shoulder, elbow and wrist is vertical, each of the lever arms can be expressed according to Equations 11 to 13. The length of the forearm f, the length of the upper arm u and the distance between the bicep insertion to the elbow b are labelled in FIG. 5. The angle θ_{H} may be an angle of the subject's hand supporting the mass 160 and the angle θ_{b} may be the angle at the bicep insertion.

$${LA}_{FA , 1} = cgf sin ( {θ}_{H} ) \qquad ( 11 )$$

$${LA}_{bal , 1} = f sin ( {θ}_{H} ) \qquad ( 12 )$$

$${LA}_{bic , 1} = b sin ( {θ}_{b} ) \qquad ( 13 )$$

The length of each lever arm can also be calculated according to Equations 14 to 16.

$${LA}_{FA , 1} = cgf sin ( {θ}_{u} - {θ}_{arm} - 90 ° ) \qquad ( 14 )$$

$${LA}_{bal , 1} = f sin ( {θ}_{u} - {θ}_{arm} - 90 ° ) \qquad ( 15 )$$

$${LA}_{bic , 1} = \frac{b sin ( {θ}_{u} )}{\sqrt{{b}^{2} + {u}^{2} - 2 bu cos {θ}_{u}}} \qquad ( 16 )$$

The angle θₐᵣₘ may be an angle of the upper arm to the horizon and the angle θᵤ may be the angle between the forearm and the upper arm.

Using these equations, the computer 105 can calculate the force experienced by the subject in real time by measuring the different angles and recalculating the force. These force calculations are made possible by calibrating the system 100 to determine the coordinates of each joint point in reality.

These equations are generally applicable for when the subject's body is positioned within the vertical plane. For subjects not entirely within the vertical plane, the program must account for the altered position as will be understood by a person of ordinary skill in the art. Based on measurements taken by the sensor 110, the computer 105 can account for these variations when outputting its final measurement (e.g., force value). For example, the expressions of lever arms in the above bicep calculation will be different.

In certain forms, the subject may use the information to inform and adjust their actions. For example, the subject may use the system 100 while lifting weights or performing another form of exercise. After calibrating the system 100, the sensor 110 may measure different forces exercised by the subject while performing that exercise. The computer 105 may output these forces in real time onto the display 115, thereby permitting the subject to view forces that their body exercise as they occur. For example, the computer 105 may make new calculations once every period so that the output is constantly updating at the expiration of each period. In some forms, a period may be between about 0.0001 seconds and about 30 seconds. In some forms, a period may be between about 0.001 seconds and about 20 seconds. In some forms, a period may be between about 0.01 seconds and about 10 seconds. In some forms, a period may be between about 0.05 seconds and about 5 seconds. In some forms, a period may be between about 0.1 seconds and about 1 second.

In certain forms, the computer 105 may compare the forces experienced by the subject against one or more threshold force values. For example, the computer 105 may be programmed with certain force values indicative of the occurrence of injuries. When the subject moves, the computer 105 may compare the forces experienced by the subject with the threshold values. If any force exceeds the corresponding threshold value, the computer 105 may provide an alert to the subject. For example, a message may be output by the display 115 (e.g., an auditory and/or visual output) intended to communicate that the subject has exceeded a threshold. This may alert the subject to stop their current activity before a potential injury occurs.

In one form, the computer 105 may output steps or suggestions for how to avoid exceeding a particular threshold. For example, the computer 105 may output, via the display 115, a message informing the subject of proper form (e.g., when running or lifting weights) to avoid creating excess force on their body.

One of ordinary skill will appreciate that the exact dimensions and materials are not critical to the disclosure and all suitable variations should be deemed to be within the scope of the disclosure if deemed suitable for carrying out the objects of the disclosure.

One of ordinary skill in the art will understand that the terms “subject,” “person,” “user,” “patient,” and/or any other similar term may be used interchangeably to describe someone who is measured by and uses the system.

One of ordinary skill in the art will also readily appreciate that it is well within the ability of the ordinarily skilled artisan to modify one or more of the constituent parts for carrying out the various embodiments of the disclosure. Once armed with the present specification, routine experimentation is all that is needed to determine adjustments and modifications that will carry out the present disclosure.

The above embodiments are for illustrative purposes and are not intended to limit the scope of the disclosure or the adaptation of the features described herein. Those skilled in the art will also appreciate that various adaptations and modifications of the above-described preferred embodiments can be configured without departing from the scope and spirit of the disclosure. Therefore, it is to be understood that, within the scope of the appended claims, the invention may be practiced other than as specifically described.

What is claimed is:

1. A method of calibrating an augmented reality system, the method comprising:
orienting a single optical sensor in a first direction, wherein the single optical sensor is connected to a computer and includes a line of sight;
outputting first instructions via a display connected to the computer, wherein the first instructions are configured to communicate to a subject to move into the line of sight and position their body in a first position;
obtaining a first measurement with the optical sensor, wherein the first measurement is a first length of a first segment of the body;
communicating the first measurement to a processor of the computer;
outputting second instructions via the display, wherein the second instructions are configured to communicate to move into the line of sight and a subject to position their body in a second position;
obtaining a second measurement with the optical sensor, wherein the second measurement is a second length of the first segment of the body;
communicating the second measurement to the processor;
calculating, via the processor, a depth based on the first measurement and the second measurement, wherein calculating includes,
determining the value of a first constant using the first measurement and the second measurement,
determining the value of a second constant using the first measurement and the second measurement,
determining the distance using the first constant and the second constant, and
storing the distance in the computer as the depth.

2. The method of claim 1, wherein the optical sensor is a webcam connected to a computer.

3. The method of claim 1, wherein calculating coordinates of the depth further includes adjusting the distance to reduce distortion by,
outputting third instructions via the display, wherein the third instructions are configured to communicate to the subject to move into the line of sight and position their body in a third position;
obtaining a third measurement with the optical sensor, wherein the third measurement is a third length of the first segment of the body;
communicating the third measurement to a processor of the computer;
calculating a distortion ratio using the third measurement and the first measurement; and
revising the distance using the distortion ratio;
wherein the distortion ratio is configured to account for an angle of inclination of the optical sensor.

4. The method of claim 1, wherein the first measurement is configured to be a first length of a first segment of the body and the second measurement is configured to be a second length of the first segment of the body.

5. The method of claim 1, further comprising,
outputting a request via the display for at least one piece of data;
receiving the at least one piece of data; and
communicating the at least one piece of data to the processor;
wherein the at least one piece of data includes a height of the subject.

6. The method of claim 5, further comprising calculating a calibration ratio using the at least one piece of data and the first measurement.

7. The method of claim 6, further comprising calculating coordinates of a tracking point by the first measurement and the second measurement, wherein the tracking point is located on the segment.

8. The method of claim 1, wherein the first instructions are configured to communicate a direction to the subject to orient their arms along a first axis, and wherein the second instructions are configured to communicate a direction to the subject to orient their arms along a second axis different than the first axis.

9. The method of claim 1, further comprising,
outputting preliminary instructions configured to communicate to the subject an initial distance to stand relative to the single sensor; and
wherein obtaining the first measurement and the second measurement are configured to confirm the initial distance.

10. A method of calculating real-time forces experienced on a subject, the method comprising:
performing the calibration steps of claim 1;
measuring a first orientation angle of the first segment using the sensor and communicating the first orientation angle to the computer;
calculating a first lever arm length using the first orientation angle;
determining a first torque using the first lever arm length; and
outputting the first force on the display;
wherein the first force is updated every period.

11. The method of claim 10, wherein a period is less than one second.

12. A method of calculating real-time forces experienced by a subject, the method comprising:
calibrating an augmented reality system to a specific subject, wherein calibrating includes,
outputting first instructions via a display connected to the computer, wherein the first instructions are configured to communicate to a subject to move into a line of sight of a sensor and position their body in a first position;
obtaining a first measurement with the sensor, wherein the first measurement is a first length of a first segment of the body;
communicating the first measurement to a processor of the computer;
outputting second instructions via the display, wherein the second instructions are configured to communicate to move into the line of sight and a subject to position their body in a second position;
obtaining a second measurement with the sensor, wherein the second measurement is a second length of the first segment of the body;
communicating the second measurement to the processor;
calculating, via the processor, a depth based on the first measurement and the second measurement;
calculating the force experienced by the subject once per period, wherein calculating includes,
measuring a first orientation angle of the first segment using the sensor and communicating the first orientation angle to the computer;
calculating a first lever arm length using the first orientation angle;
determining a first force using the first lever arm length; and
outputting the first force on the display.

13. The method of claim 12, further comprising,
outputting a request via the display for at least one piece of data;
receiving the at least one piece of data;
communicating the at least one piece of data to the processor; and
using the at least one piece of data in the force calculation;
wherein the at least one piece of data includes a weight held by the subject.

14. An augmented reality system comprising:
a computer having a processor;
an imaging sensor connected to the computer and configured to transmit measurements to the computer;
a display connected to the computer and configured to output data from the computer;
wherein the computer is configured to implement a diagnostic program, the diagnostic program includes,
outputting an initial instruction via the display configured to instruct a subject to stand an initial distance from the imaging sensor,
orienting the imaging sensor in a first direction, wherein the imaging sensor includes a line of sight
outputting a first calibration instruction via the display configured to instruct the subject to move into the line of sight and orient their arms in a first position,
receiving, from the imaging sensor, a first measurement following delivery of the first calibration instruction, wherein the first measurement is a first length of a first segment of the body,
outputting a second calibration instruction via the display configured to instruct the subject to move into the line of sight and orient their arms in a second position,
receiving, from the imaging sensor, a second measurement following delivery of the second calibration instruction, wherein the first measurement is a first length of a first segment of the body,
calculating, using the first measurement and the second measurement, a depth, wherein calculating includes,
determining a value of a first constant using the first measurement and the second measurement,
determining a value of a first constant using the first measurement and the second measurement,
determining the distance using the first constant and the second constant, and
storing the distance in the computer as the depth.

15. The augmented reality system of claim 14, wherein the imaging sensor is a single imaging sensor.

16. The augmented reality system of claim 14, wherein the diagnostic program further includes,
outputting a third calibration instruction via the display configured to instruct the subject to orient their arms in a third position,
receiving, from the imaging sensor, a third measurement following delivery of the second calibration instruction,
calculating a distortion ratio using the first measurement and the third measurement, and
adjusting the first measurement using the distortion ratio to account for the orientation of the imaging sensor.

17. The augmented reality system of claim 14, wherein the data is a length measurement configured to correspond to the subject's height.

18. The augmented reality system of claim 14, wherein the first calibration instruction is configured to communicate a direction to the subject to orient their arms along a first axis, and wherein the second calibration instruction is configured to communicate a direction to the subject to orient their arms along a second axis different than the first axis.

19. The augmented reality system of claim 14, wherein the diagnostic program further includes,
calculating the coordinates of the joints of a body of the subject in reality,
calculating the lengths of the segments of a body of the subject in reality,
calculating the angles among the segments when a body of the subject is at different static gestures and in motion.

20. The augmented reality system of claim 14, wherein the diagnostic program further includes,
calculating one or more real-time forces exerted on the subject.
